(19) United States
(12) Patent Application Publication
(10) Pub. No.: US 2011/0150995 A1
(43) Pub. Date: Jun. 23, 2011

(54) Solid Dosage Forms of Essential Oils

(76) Inventor: Hemant Narahar Joshi, Parsippany, NJ (US)

(21) Appl. No.: 12/645,446

(22) Filed: Dec. 22, 2009

(51) Int. Cl. A61K 9/28; A61K 9/00; A61K 36/61

(52) U.S. Cl. 424/474; 424/484; 424/742

(57) ABSTRACT

Essential oils are extracted from plants and some have known medicinal properties. Some essential oils have been to known to show antibacterial, antifungal and antiviral activities. Some essential oils show sensual properties and others improve quality of sleep. The present invention describes a method to produce a solid dosage form (tablets or capsules) of these essential oils showing medicinal properties. The essential oils are adsorbed or absorbed on a substrate. The presence of a surfactant in the formulation helps solubilization of essential oils in the mouth cavity in vivo. Combination of various essential oils can be incorporated in the solid dosage forms.

### FIELD OF INVENTION

The present invention relates to the preparation of solid dosage forms (tablets or capsules) of essential oils. Essential oils are volatile, hydrophobic potent liquids derived from plants. Many essential oils have useful pharmacological activities and are very potent. Some essential oils are known to have antibacterial, antifungal and antiviral activities. Some essential oils have sensual properties and some may help treat sleep disorders. The present invention will allow patients to take small doses of essential oils in a single dosage form. The present invention relates to solid dosage forms containing essential oils for treating, killing, and/or inhibiting growth of fungal, viral and bacterial pathogens in human subjects, or to solid dosage forms containing essential oils having sensual properties or improving sleep disorders.

### BACKGROUND OF THE INVENTION

Essential oils, also known as volatile oils, are hydrophobic, concentrated liquids derived from plants. Each essential oil has a characteristic aroma or fragrance. They are used in perfumes, bath product, as flavoring agents in foods and drinks, in household cleaning products. Some essential oils also have medicinal/therapeutic properties. Essential oils showing medicinal value are currently being used in aromatherapy.

Essential oils are extracted from plants by distillation, expression or solvent extraction methods. Various parts of plants, such as seeds, berries, barks, wood, rhizome, leaves, flowers, and roots are used as the source of essential oils.

In general, essential oils are very potent as they are in a concentrated form and there are few safety concerns. Thus, care must be taken during their consumption, especially if used for their therapeutic value. Some essential oils have a low flash point (flash point is the lowest temperature at which a substance can vaporize to form an ignitable mixture in air). Because essential oils are plant derived, there could be some concern about the presence of pesticides, herbicides, etc. and care must be taken during their extraction. A person consuming these essential oils should look for any allergic reactions. The amounts of essential oils consumed per day must be controlled. Some of these essential oils should not be consumed during pregnancy.

Some essential oils show antiviral, antibacterial, and antifungal activities. Viruses are infectious agents carrying genes on their DNA or RNA surrounded by a protein coat. Some viruses are enveloped by a fatty coat. Viruses can replicate inside the cells of other organisms. The scare of H1N1 virus causing the swine flu in recent times has developed awareness for different antiviral treatments. Previously, SARS (severe acute respiratory syndrome), caused by a pneumonia-like virus, created havoc. Common flu or influenza viruses are very well-known, too. There are very few targets in viruses on which drugs can attack and as a result, effective antiviral drugs are limited. Our immune system normally can kill the invading viruses. A lot is known about bacteria and fungi. Many antibacterial and antifungal agents are already in use. However, bacteria have developed resistance to antibiotics and newer treatments are always being sought.

Medicines derived from plant sources have been used by humans for centuries. U.S. Pat. No. 6,264,926 claimed formulations of herbal toothpowder or toothpaste for gums and teeth. These contained a powder form of different plants as well as a few essential oils including eucalyptus and clove oils. U.S. Pat. No. 6,589,513 described the usage of herbal extracts and essential oils employing mouthwash, toothpaste, toothbrush bristles, floss and ointment as the delivery systems. U.S. Pat. No. 6,379,652 described various formulations such as mouthwash solutions, gels, toothpastes containing essential oils including tea tree oil, thymol, eugenol, eucalyptus oil and spearmint oil along with menthyl ester (menthol reacts with carboxylic acids to produce menthyl esters). Menthyl ester was reported to enhance the antibacterial activity of essential oils. U.S. Pat. No. 7,011,093 demonstrated the use of cosmetic fragrances and aromatic medications in the form of nasal strips. The aromatic medication contained camphor, eucalyptus oil, peppermint oil, menthol, methyl salicylate, bornyl acetate and lavender oil. In the U.S. Pat. No. 6,607,778, the inventors prepared solid delivery system of maltodextrin and at least one material selected from the group consisting of sucrose, glucose, lactose, levulose, maltose, fructose, isomalt, sorbitol, mannitol, xylitol, lactitol, maltitol and hydrogenated corn syrup as a mixture and incorporated aroma chemicals and flavoring agents. This extrusion system is used to flavor consumer products. U.S. Pat. No. 6,607,771 described the process of preparation of granules for the controlled release of volatile compounds. The encapsulated aromatic compounds in the granules would be released in a controlled fashion in food products and also, prevent their losses during storage. U.S. Pat. No. 6,444,253 described an anhydrous liquid flavor delivery system, which has three main components—a mixture of flavors, surfactants and alcohol composition.

The following types of active ingredients are found in essential oils demonstrating antimicrobial activities—monoterpenes, sequiterpenes, diterpenes, terpene alcohols, phenols, aldehydes, ketones, esters, and oxides. Some essential oils also show immune-stimulating properties. Echinacea is considered as a “blood purifier” due to its anti-inflammatory and immune stimulating properties. Goldenseal, a native of North America, has been used for its antibiotic, anti-inflammatory and astringent properties. It is taken in herbal medicine at the first signs of respiratory problems, colds and flu. German researchers have reported anti-viral activity of Echinacea against influenza pathogens. The following are the reported combinations of essential oils used to treat various diseases—sore throat: thyme and marjoram oil, common cold: eucalyptus oil, asthma: lavender, chamomile, atlas cedarwood, geranium pine, eucalyptus and frankincense, pneumonia: thyme, oregano, and rosewood, bronchitis: fir, eucalyptus, lavender, myrtle, pine, hyssop and spruce, sinus infection: tea tree oil, eucalyptus, and peppermint.

The Ananda Apothecary reported the following essential oils to have antiviral activity: Melissa, Ravensara, Bay Laurel, Tea Tree, Frankincense, Eucalyptus radiate, Hyssop, and Lavender. Melissa, a lamiaceae plant, was shown to be efficacious against the herpes virus. In Germany, a cream containing an extract of Melissa is sold to treat herpes. Schnitzler et al. (Pharmazie 56:343-347, 2001) reported an antiviral effect of Australian Tea Tree oil and eucalyptus oil against herpes simplex virus. Khafagi et al. studied the antibacterial and anticandidal activity of seven commonly used essential oils. The oils of clove showed a broad spectrum antimicrobial activity (Egyptian J. Biol. 2:20-27, 2000). Pasqua et al. investigated antimicrobial activity of twelve essential oils against gram positive and gram negative bacteria, for potential use in food industry. All the oils showed satisfactory antimicrobial activity and authors recommended to use these oils for food preservation (Annals Microbiol. 55, 139-143, 2005).

Hyssop is from the family lamiaceae, which is native to East Mediterranean region. It is referred in the Bible and was used in salads, soups and in meat. Cajeput oil helps to clear nasal passages when used in steam inhalation and it has antiseptic properties. Frankincense oil has been burnt for centuries on altars and in temples. It creates a spiritual atmosphere and controls tension. Helichrysum oil helps to clear the mind, chest and sinus. Juniper oil is reputed to strengthen the immune system. Lavender oil is the most versatile essential oil and there is a saying that NO home should be without it. Lemon oil has antiseptic and soothing properties. Manuka oil is antibacterial and antifungal and is particularly effective against staphylococcus and streptococcus infections. Sweet marjoram oil helps to relieve congestion due to common cold and it has antiseptic properties. Myrrh oil has antiseptic and anti-inflammatory properties. Niaouli oil has been used locally for respiratory ailments, body aches and pains, and infections. It has been reported to activate the immune system. HaEmek Medical Center, in Israel, recently completed a study to understand the effect of essential oils on the treatment of acute pharyngo-tonsillitis (Harefuah. 147:783-788, 838 (2008)). They used a 3% mixture of aromatic essential oils containing Eucalyptus citriodora, Eucalyptus globules, Mentha piperita, Origanum syriacum, and Rosmarinus officinalis as a solution. This formulation has an activity on the respiratory tract, the coughing reflex, and the airflow in the nasal tract.

Based on the empirical evidence and scientific studies, some of the essential oils have medicinal properties and can be used to treat diseases. A solid dosage form, which is easy to administer, will have a high clinical value.

### SUMMARY OF INVENTION

Some of the essential oils show important pharmacological activities. Some are known to have antibacterial, antifungal and antiviral activities. Others have sensual properties and some are effective against sleep disorders. These essential oils are very potent and should be used in small doses. A right combination of essential oils could be more effective to treat these ailments. Solid dosage forms, mainly tablets and capsules, providing therapeutically effective amounts of essential oils are not available in the market. The main objective of this invention is to provide solid-dosage forms (tablets or capsules) of essential oils. It is an objective of the present invention to absorb or adsorb the essential oils on a solid matrix, which is subsequently mixed with other ingredient to form the solid dosage form. It is another object of the present invention to allow solubilization of essential oils in the oral cavity with the help of a surfactant and subsequently enhance their vaporization. Being hydrophobic, the essential oils have low solubility in the aqueous environment. Thus, the addition of suitable surfactant/surfactants would help to solubilize essential oils in the mouth cavity. The vapors and the solubilized fraction of these essential oils can act in the mouth, in the throat area and could be inhaled into the lungs. Thus, the chewable or orally disintegrating tablets will release these essential oils in the mouth cavity for local action. The immediate release, controlled release or enteric-coated tablets and capsules will release the essential oils in the gastro-intestinal tract with help of the surfactant/surfactants, where they can be absorbed easily. The tablets or capsules may contain one essential oil as an active ingredient or may contain a combination of two or more essential oils.

The following essential oils are known to have antibacterial, antiviral and antifungal activities and can be incorporated in the chewable or orally disintegrating tablets. These are—ajowan, aniseed, atlas cedarwood, bay laurel, bergamot, cajeput, caraway, chamomile, clove, cumin, cypress, echinacea, elemi, eucalyptus, fennel, fir, frankincense, goldenseal, grapefruit, helichrysum, hyssop, juniper, kanuka, lavender, lemon, lemongrass, manuka, marjoram, melissa, myrrh, myrtle, niaouli, nutmeg, orange, oregano, peppermint, petitgrain, pine, ravensara, rose geranium, rosewood, sage, savory, spruce, tea tree, thyme, valerian, verbena, and vervain.

Another object of the present invention is to incorporate essential oils which are known to be aphrodisiacs and have sensual properties. A chewable or orally disintegrating tablet of these oils would release the actives in the oral cavity. The examples of such oils are—cinnamon, elemi, fennel, jasmine, sandlewood, vetiver and ylang ylang oil.

Another object of the present invention is to incorporate essential oils, which are known to improve quality of sleep. These essential oils are known to calm mind and help it to relax. Examples of essential oils with such properties are—cajeput, clary sage, lavender, niaouli, and nutmeg oil. Administration of chewable or orally disintegrating tablets with such essential oils prior to going to bed would help improve quality of sleep.

Another object of the present invention is to incorporate sodium chloride in the formulation. Sodium chloride is known to have antibacterial activity and it can provide appealing taste to the chewable and orally disintegrating tablets.

Some of these essential oils are broad-spectrum being effective against different ailments, for example, eucalyptus and lavender oil. Thus, tablets of such essential oils can be used for different therapeutic values.

### DETAILED DESCRIPTION

Different essential oils have antibacterial, antifungal and antiviral activities. Few clinical studies have been conducted to examine the activity of each essential oil against various diseases. However, a general empirical knowledge has proven the effectiveness of essential oils against many sets of bacteria, viruses and fungi. Each essential oil contains several active ingredients and they may be effective against different organisms. These essential oils are found to be effective when used alone. However, in many instances, a combination of essential oils in the treatment has been observed to be more effective.

This invention includes development of solid dosage forms of essential oils which have been known to show antibacterial, antiviral and antifungal activities. The invention also includes solid dosage forms of other essential oils known to have aphrodisiac/sensual properties and improve sleep quality. The solid dosage form may encompass tablets or capsules. The tablets could be used as “orally disintegrating”, “immediate release” or “chewable” and could be “enteric coated”. In the chewable and orally disintegrating tablets, the essential oils will dissolve in saliva in the presence of a suitable surfactant and act in the oral cavity, in the throat or they will vaporize to be inhaled into the lungs. In the immediate release tablets, the essential oils will be released in the gastrointestinal tract. The tablets could be “enteric coated” and would release the essential oils in the intestine after bypassing the stomach. The capsule dosage form will release the actives in the stomach.

The tablets include the following key ingredients—essential oils, adsorbents, diluents, binders, and lubricants. Other commonly used ingredients such as flavoring agents, coloring agents, glidants, coating agents, taste-masking agents can also be included.

The following essential oils are known to have antibacterial, antiviral and antifungal activities and can be incorporated in the tablets. These are—ajowan, aniseed, atlas cedarwood, bay laurel, bergamot, cajeput, caraway, chamomile, clove, cumin, cypress, echinacea, elemi, eucalyptus, fennel, fir, frankincense, goldenseal, grapefruit, helichrysum, hyssop, juniper, kanuka, lavender, lemon, lemongrass, manuka, marjoram, melissa, myrrh, myrtle, niaouli, nutmeg, orange, oregano, peppermint, petitgrain, pine, ravensara, rose geranium, rosewood, sage, savory, spruce, tea tree, thyme, valerian, verbena, and vervain.

Some essential oils are known to be aphrodisiacs having sensual properties. The examples of such oils are—cinnamon oil, elemi oil, fennel oil, jasmine oil, sandlewood oil, vetiver oil and ylang ylang oil. Vetiver oil blends well with lavender, jasmine and sandlewood oils.

Some essential oils are known to calm the mind and help it to relax. Administration of tablets with these essential oils prior to going to bed would help improve quality of sleep. Examples of essential oils with such properties are—cajeput, clary sage, lavender, niaouli, and nutmeg oil. Few drops of lavender oil on the pillow and sheet helps get peaceful sleep. Niaouli oil with lavender oil is reported to be effective in treating insomnia. Nutmeg oil is used in the treatment of many ailments including indigestion, flatulence, nervous fatigue and anxiety.

In the solid dosage forms referred in this invention, during the granulation step, the essential oils are adsorbed or absorbed on a solid matrix and then mixed with other excipients. With this, the blend does not feel oily and when the tablets are compressed, the essential oils do not ooze out. Adsorbents are chemically inert materials that have capability to adsorb gases, chemicals, toxins and bacteria. Kaolin and activated charcoal are used for detoxification or removal of gases from the gastro-intestinal tract. Pectin is used to treat diarrhea. In the case of essential oils, if the absorbant matrix is porous, the oil may penetrate the pores. The most commonly used adsorbents/absorbants for oils are as follows—silicone dioxide, activated charcoal, talc, calcium silicate, maltodextrin, magnesium aluminum silicate, clay, natural starches, starch derivatives, and cyclodextrin. Based on the properties of essential oil, an appropriate adsorbent is selected. DRY-FLO PURE (Aluminum Starch Octenylsuccinate) and DRY-FLO AF (Aluminum free modified corn starch) are hydrophobically modified polymers with high oil absorption capacity. They are currently used in several topical formulations. Pregelatinized starch is commonly used in pharmaceutical dosage forms.

The diluents, binders and lubricants to be used in these solid dosage forms are known to pharmaceutical formulators who work in this field.

The tablets of essential oils may release the actives in the oral cavity or in the gastrointestinal (GI) tract. The capsules will not release the powder in the oral cavity and will release the essential oils in the stomach. In the GI tract, the bile acids would help digest the essential oils and subsequently, facilitate their absorption. However, for the chewable or orally disintegrating tablets, the intention will be to release the active oils in the oral cavity. In the oral cavity, it is difficult to dissolve these hydrophobic essential oils because they are in a very hydrophilic environment. Thus, the use of an appropriate surfactant in the formulation will help to dissolve the oils in saliva, which, in turn, will help to make the essential oils effective in the oral cavity. The micelle formation or emulsification increases the surface area of the essential oil, thereby, helping it to vaporize. The vapors can thus reach into the pulmonary tract and deep in lungs. Persons working in the pharmaceutical formulation field are aware of several surfactants commonly used in the oral dosage forms.

Sodium chloride can be used an active component in the tablets. It is known to have antibacterial property and it can provide a desirable taste to the chewable and orally disintegrating tablets.

The following table shows the composition of a tablet of essential oils, which can release the oils in the mouth cavity (chewable tablets). The manufacturing process involves the following steps: the adsorption of essential oils on the adsorbant (calcium silicate in this example) followed by mixing of a sweetening agent (aspartame), a surfactant (sodium lauryl sulfate), binder (gum arabica) and then the final mixing with the diluent (sorbitol). The tablets are compressed on a commonly used tablet press. Sodium chloride and citric acid will impart a good taste to the tablet. Sodium chloride will also act as an antibacterial agent. Weight of each tablet could vary depending upon its usage. Tablets may weigh from 100 mg to 1 g per unit.

| Ingredient | Weight, mg per 1000 mg |
|---|---|
| Sodium chloride | 50 |
| Eucalyptus oil | 10 |
| Peppermint oil | 10 |
| Clove oil | 10 |
| Calcium silicate | 50 |
| Gum Arabica | 20 |
| Sodium lauryl sulfate | 5 |
| Sorbitol | 810 |
| Aspartame | 10 |
| Citric acid | 15 |
| Magnesium stearate | 10 |
| Total | 1000 |

In another formula, tea tree oil, eucalyptus, and peppermint can be mixed to prepare tablets, which can be used to treat sinus infections.

In another formula, lavender, chamomile, atlas cedarwood, geranium pine, eucalyptus and frankincense can be delivered in a tablet to be used to treat asthma.

Essential oils are natural ingredients. They contain many active ingredients. One has to be very cautious while using these tablets. The consumer has to ensure that he/she is not allergic to the active essential oil/oils in the tablet formulation. Few oils which should not be consumed during pregnancy are—atlas cedarwood, basil oil, rosemary oil, and thyme. One must take the solid dosage form of essential oils under strict medical supervision and should report adverse reactions.

Essential oils must be purchased from the best source that produces these oils using current good manufacturing practices. The plant parts used for the extraction of essential oils must be devoid of pesticides, herbicides and other ingredients used during the cultivation of these plants. They should be devoid of heavy metal contamination too. Care must be taken to completely remove the extraction solvents from the essential oils. Extraction solvents known to be harmful to the human body should not be used. Also, precautions must be taken to prevent degradation of active ingredients during extraction processes such as distillation.

Essential oils are known to be potent and therefore, care must be taken to avoid excessive usage of these oils, especially by the oral route. Due to lack of extensive documented clinical studies, the exact dose of each essential oil for oral administration is not known. It is advisable to start with a low dose and then increase it incrementally. For the same reason, the 100 mg tablets made from the example shown above would contain only a total of 3 mg of total essential oils. The amount of each essential oil per gram of blend should be between 0.1 mg and 50 mg, preferably between 0.1 mg and 20 mg and most preferably, between 0.1 mg and 10 mg. The ratio of essential oil to adsorbent/absorbant would vary based on properties of essential oils and the adsorbent/absorbant chosen in the formulation. For calcium silicate used in the representative formulation, the desired ratio is 1:1 (w/w) to 1:5 (w/w) and the most desired ratio is 1:1 (w/w) to 1:2 (w/w).

What is claimed is:

1-8. (canceled)

9. A Process of making a solid dosage form composition of essential oils comprising the steps of: i) mixing therapeutically effective amount of essential oil or a mixture of essential oils on adsorbents or absorbents; ii) mixing the mass from Step i with a surfactant; iii) mixing the mass from Step ii with excipients of solid dosage forms, wherein the excipients are diluents, binders, coloring agents and lubricants; and iv) converting the mixture from Step iii to a solid dosage form; wherein the solid dosage form is a tablet or a capsule.

10. A Process of making a solid dosage form composition of essential oils according to claim 9 wherein the essential oil or a mixture of essential oils have a therapeutic activity.

11. A Process of making a solid dosage form composition of essential oils according to claim 9 wherein the adsorbent or absorbent is selected from the group comprising of calcium silicate, silicone dioxide, magnesium aluminum silicate, and mixtures thereof.

12. A Process of making a solid dosage form composition of essential oils according to claim 9 wherein the tablet is selected from the group comprising of chewable, orally disintegrating, immediate release, controlled release or enteric coated tablets.

13. The essential oils having a therapeutic activities in claim 10 wherein the essential oils incorporated are selected from the group comprising: ajowan, aniseed, atlas cedarwood, bay laurel, bergamot, cajeput, caraway, chamomile, cinnamon, clary sage, clove, cumin, cypress, echinacea, elemi, eucalyptus, fennel, fir, frankincense, goldenseal, grapefruit, helichrysum, hyssop, jasmine, juniper, kanuka, lavender, lemon, lemongrass, manuka, marjoram, melissa, myrrh, myrtle, niaouli, nutmeg, orange, oregano, peppermint, petitgrain, pine, ravensara, rose geranium, rosewood, sage, sandlewood, savory, spruce, tea tree, thyme, valerian, verbena, vervain, vetiver, ylang ylang oil and mixtures thereof.
